(19) United States
(12) Patent Application Publication
(10) Pub. No.: US 2026/0337149 A1
(43) Pub. Date: Oct. 29, 2026

(54) SYNTHESIS OF NON-IONIC RADIOGRAPHIC CONTRAST AGENTS BY MEANS OF REACTIVE EXTRUSION

(71) Applicant: BRACCO IMAGING S.P.A., Milan (IT)

(72) Inventors: Luciano LATTUADA, Cassina De'Pecchi (IT); Roberto SEBASTIANO, Lazzate (IT); Gabriella LEONARDI, Milano (IT); Giancarlo CRAVOTTO, Torino (IT); Alessandro BARGE, Torino (IT); Fabio BUCCIOL, Rivoli (IT)

(73) Assignee: BRACCO IMAGING S.P.A., Milan (IT)

(21) Appl. No.: 19/491,122

(22) PCT Filed: Jun. 10, 2024
(86) PCT No.: PCT/EP2024/065899

§ 371 Date: Dec. 8, 2025

(30) Foreign Application Priority Data
Jun. 13, 2023 (EP) 23178956.1

(51) Int. Cl. C07C 231/12

(52) U.S. Cl. CPC C07C 231/12

(57) ABSTRACT

The present invention relates to the industrial preparation of non-ionic X-ray contrast agents. In particular, it relates to a process for the synthesis of lopamidol using a reactive extrusion process in continuous which enable an efficient conversion of the relevant key intermediates, preferably in the absence of any solvent. The invention further relates to the preparation of radiographic X-rays contrast agents or key intermediates thereof by exploiting the technology of reactive extrusion.

### FIELD OF THE INVENTION

The present invention relates to the industrial preparation of non-ionic X-ray contrast agents. In particular, it relates to a process for the synthesis of Iopamidol using a reactive extrusion process in continuous, which enable an efficient conversion of the relevant key intermediates, preferably in the absence of any solvent. The invention further relates to the preparation of radiographic X-rays contrast agents or key intermediates thereof by exploiting the technology of reactive extrusion.

### BACKGROUND OF THE INVENTION

Iodinated contrast agents are well-known compounds widely used in X-ray imaging diagnostic techniques. Among these compounds, S—N¹,N³-bis[2-hydroxy-1-(hydroxymethyl)ethyl]-5-[[(2S)-2-hydroxy-1-oxopropyl]-amino]-2,4,6-triiodo-1,3-benzene dicarboxamide (Iopamidol) is a radiographic contrast agent widely used in daily diagnostic investigations by X-rays (The Merck Index, RSC Publishing, 15^{th} Ed., 2013, 940-941; Lusic, H. et al., Chem. Rev. 2013, 113, 1641-1666). This compound can be prepared by using a synthetic procedure known since the eighties and disclosed e.g. in GB 1,472,050. An industrial synthesis of Iopamidol is also shown in the following Scheme 1 and described, for example, in U.S. Pat. No. 4,001,323.

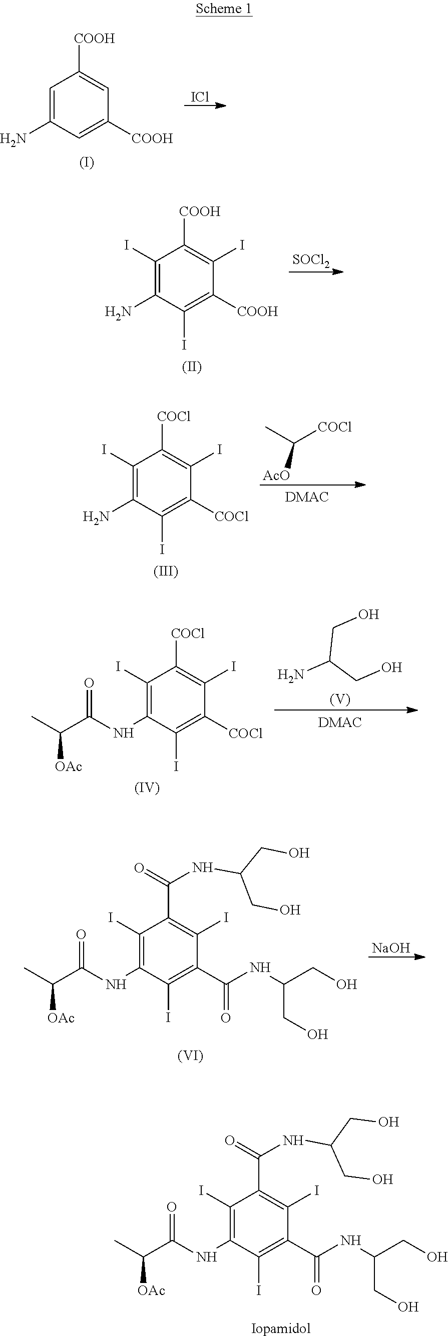

The industrial process currently used for the manufacturing of Iopamidol comprises the amidation reaction of S-5-[[2-(acetyloxy)-1-oxopropyl]amino]-2,4,6-triiodo-1,3-benzenedicarboxylic acid dichloride (IV) with 2-amino-1,3-propanediol (commonly known as serinol, V) to obtain Acetyl Iopamidol (VI), which is further hydrolyzed to obtain Iopamidol. Such reaction is typically carried out in dipolar aprotic solvents, e.g. N,N-dimethylacetamide (DMAC) or N,N-dimethyformamide (DMF), which represent an optimal solution in order to solubilize both the lipophilic aryl intermediate (IV) and the hydrophilic hydroxy-alkyl amine (V), thus facilitating their interaction.

However, such solvents suffer from the disadvantage of being reprotoxic and flagged under the “Registration, Evaluation, Authorization and restriction of Chemicals” (REACH) legislation as Substances of Very High Concern, especially when they are used in the bulk volumes of an industrial production. The regulators are more frequently inviting to the use of sustainable and environmentally friendly solutions when performing chemical reactions in the pharmaceutical industry and many efforts are being performed in this direction, in particular with the aim of reducing or eliminating hazardous substances. Moreover, avoiding the use of toxic solvents not only decreases potential hazards, but can also reduce the overall costs of a process, including costs for waste treatment.

Other aprotic dipolar solvents, such as N-methylpyrrolidone (NMP) or N-ethylpyrrolidone (NEP), tested with good results in these reactions (see for instance GB 2,311,524), are equally reprotoxic, therefore not suitable to solve this safety issue. Moreover, they display a higher boiling point and they are difficult to be removed from the products at the end of the reactions and from the bulk drug substance, especially in an industrial scale process.

One approach towards the replacement of undesirable polar aprotic solvents has been described for instance in WO00/15602, where it is disclosed a process for the preparation of acetyl Iopamidol by reaction of compound (IV) with serinol (V) under heating and in the absence of any solvent and any base. To obtain good conversions, however, it was necessary to use a very high excess of serinol, with molar ratio between serinol (V) and compound (IV) ranging from 6 up to 25.

Alternatively, mechanochemical approaches were considered, which are generally conducted by grinding together two or more solid reagents to instigate a chemical reaction. One relevant example is reported in WO2018/104228 which discloses a mechanochemical process exploiting the use of mechanical milling of specific reactants for the manufacturing of key intermediates of radiographic contrast agents, like compound (VI), without providing any external heating and substantially without the addition of solvents.

However, this approach suffers for limited scalability, in particular for safety reasons. In fact, the use of larger ball mills, suitable for processing high amounts (e.g. kilograms) of products for industrial manufacturing, has never been demonstrated for chemical syntheses. Moreover, the mechanochemical approach can be carried out in batch mode only and, in some cases, it can have the drawback of a limited temperature control.

As a consequence, there is an increasing need for a more sustainable process for the preparation of Iopamidol and the intermediates thereof, providing a safer and efficient alternative to the use of toxic solvents which should be also applicable at industrial scale with good productivity results.

Recently a great industrial interest has grown up for the application of reactive extrusion techniques in the manufacturing of compounds, as possible efficient alternative to classical chemical processes or to other processes exploiting mechanical means (e.g. by grinding and milling neat reactants) providing solventless, continuous and scalable methods.

Reactive extrusion (REX) is a well-known technology referring to a family of continuous processing techniques, i.e. manufacturing methods combining the traditional chemical processes (syntheses and/or modifications, typically of polymeric materials) and extrusion (pushing materials through a die by applying compressive and shear forces) into a single process carried out onto an extruder.

A typical continuous equipment (extrusion line) consists of at least a feeder (volumetric or gravimetric dosing system) and a barrel, wherein the materials are intensively mixed and forced through constrained spaces. Such barrel can be heated and can contain either one or more screws that convey the materials along the inside of the barrel and subject it to shearing and mixing forces before the exit. Several extrusion parameters are generally tuned to optimize the processes, including screw speed, screw profile, feed rate, residence time and temperature. An extruder can be also integrated into a continuous manufacturing process as either one component of a processing line or as the entire line itself.

Reactive extrusion is scalable up to tons of product per hour, allowing reactions to be carried out with little or no solvent. For this reason, the extrusion technology has been employed across several industries, including food, polymer and pharmaceutical manufacturing (mostly in the formulation of drugs). Most applications of reaction extrusion techniques have been described to produce polymers of high molecular weight, for instance in the manufacturing of chemically modified natural macromolecules or synthetic polymers.

Conversely, extrusion approaches have not been yet extensively applied to the preparation of active pharmaceutical ingredients (APIs). In fact, only very recently the use of the extrusion technique has been explored in other fields, such as in the synthesis of organic compounds through reactions like Knoevenagel condensations, Michael additions and aldol reactions, often without the need of post-synthetic isolation or additional purification. Examples of these methods are reported in Crawford D. E. et al, Green Chem., 2017, 19, 1507 and in Crawford D. E. et al, Chem. Commun. 2017, 53, 13067-13070.

Reactions of amidation by reactive extrusion for the synthesis of active pharmaceutical ingredients teriflunomide and moclobemide are described in Lavayssiere M. et al, Chem. Commun. 2023, 59, 3439-3442 using a vertical conical extruder. The amidation reactions on these compounds were performed in batch with a recirculation system and using a coupling reagent like CDI, EDC·HCl and COMU in the presence of a liquid additive and preferably a base.

Examples disclosing the use of an extruder for preforming organic chemistry reactions in continuous are represented by patents U.S. Pat. No. 5,859,269 describing a process for the continuous production of monocarboxylic acids from the corresponding alcohols wherein the oxidation reaction is continuously accomplished in an extrusion reactor; U.S. Pat. No. 5,859,263 describing a continuous process for preparing levulinic acid from starch in a reactive extrusion process; and U.S. Pat. No. 9,453,107 describing the preparation of a polyamide from selected monomer(s) which are introduced in an extruder without any preliminary treatment.

Notwithstanding the above disclosures, further developments are needed to assess the possible application of reactive extrusion for the preparation in continuous of different classes of organic products and intermediates. In fact, each molecule can display different features in terms of reactivity, solubility, state of matter based on temperature and pressure (solid, liquid, gas), rheological behavior, viscosity and the like, so that an optimization of specific operative conditions, such as for instance temperature and/or rotation speed of the screws, is necessary for different compounds and types of reaction. Moreover, the scaling up of the reactions in an industrial perspective is not always viable and simple, for instance due to the design and cost of the necessary equipment.

Surprisingly, it has been found that a reactive extrusion process can be efficiently exploited for the preparation of a radiographic contrast agent, such as Iopamidol, and in particular to carry out the above described amidation in continuous of compound (IV) with serinol (V). According to the present invention, compounds (IV) and (V) are employed as the sole reactants which are continuously fed and mixed in the reaction chamber of an extruder, thereby interacting while being transported to the outlet of the reaction chamber itself, in order to provide the intermediate acetyl-Iopamidol (VI), partially in admixture with Iopamidol, which can be then hydrolyzed to afford the final product Iopamidol.

The process of the invention was also found scalable and suitable for an industrial manufacturing of Iopamidol, and more generally of radiographic contrast agents.

### SUMMARY OF THE INVENTION

The present invention generally relates to an industrial preparation of the non-ionic X-ray contrast agent Iopamidol, providing a sustainable method that avoids the use of toxic and highly boiling solvents. In particular, according to the invention, Iopamidol can be obtained through a continuous process exploiting the reaction extrusion technology for the interaction and conversion of its key intermediates.

In fact, it was unexpectedly found that, in accordance with the present invention, extrusion processing of a slurry comprising S-5-[[2-(acetyloxy)-1-oxopropyl]amino]-2,4,6-triiodo-1,3-benzenedicarboxylic acid dichloride (IV) in admixture with 2-amino-1,3-propanediol (V) provides a very efficient method for the production of Iopamidol, without using any hazardous solvent, since the reaction extrusion of the invention is performed in continuous by directly feeding the reactants (IV) and (V) in a suitable extruder.

Accordingly, in one aspect the present invention relates to a new process in continuous for the manufacturing of the radiographic agent Iopamidol which exploits the use the technique of reaction extrusion to perform the amidation of S-5-[[2-(acetyloxy)-1-oxopropyl]amino]-2,4,6-triiodo-1,3-benzenedicarboxylic acid dichloride (IV) with 2-amino-1,3-propanediol (V), obtaining the intermediate acetyl-Iopamidol (VI) in admixture with an amount of Iopamidol, according to the following Scheme 2:

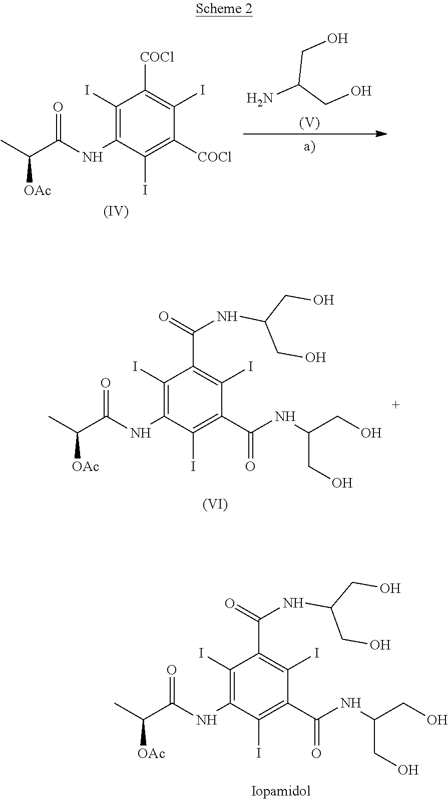

The residual amount of intermediate (VI) is then all converted into the final radiographic agent Iopamidol by hydrolysis of the acetyl group in basic aqueous conditions.

The extrusion process of the invention (step a) is advantageous with respect to the methods described in the prior art in that the process is operated in continuous, requires few steps and reduced reaction times and is more sustainable and economic since it is substantially solventless.

In particular, the process of the invention allows to obtain the final product Iopamidol with a good efficiency and without using any toxic material, such as the solvent DMAC or DMF which are under strict restrictions by the REACH Directive. In fact, the process of the invention can provide conversions of compound (IV) up to 96% and final yields of Iopamidol after hydrolysis higher than 70%, in very short reaction times.

Moreover, the process of the invention is of general applicability and provides a synthetic approach generally exploitable for the preparation of radiographic contrast agents and/or their relevant key intermediates.

It is yet another object of the invention to provide a process in continuous for the preparation of Iopamidol by reactive extrusion wherein the excess of serinol (V) contained in the extruded stream is recovered and recycled in the process.

### BRIEF DESCRIPTION OF THE DRAWINGS

The objects and advantages of the present invention may be better understood by those skilled in the art by reference to the accompanying figure, wherein:

FIG. 1 shows a schematic representation of a reactive extruder equipped with one screw and one inlet section according to a preferred embodiment of the present invention.

FIG. 2 shows a schematic representation of a reactive extruder equipped with two co-rotating screws and two inlet sections according to another preferred embodiment of the present invention.

### DETAILED DESCRIPTION OF THE INVENTION

In one aspect the invention relates to a process for the preparation of N¹,N³-bis[2-hydroxy-1-(hydroxymethyl)ethyl]-5-[[(2S)-2-hydroxy-1-oxopropyl]amino]-2,4,6-triiodo-1,3-benzenedicarboxamide (Iopamidol) comprising the following steps:
a) mixing and reacting S-5-[[2-(acetyloxy)-1-oxopropyl]amino]-2,4,6-triiodo-1,3-benzenedicarboxylic acid dichloride (IV) with 2-amino-1,3-propanediol (V) thus obtaining a mixture of the intermediate acetyl-Iopamidol (VI) and Iopamidol

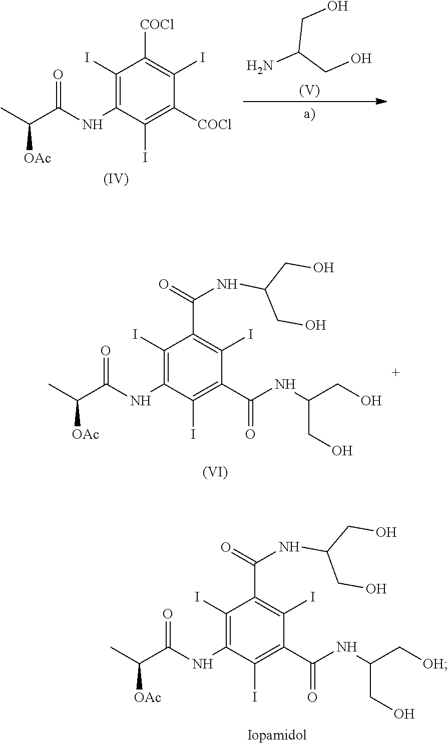

b) treating the mixture thus obtained in basic aqueous conditions by promoting the removal of the acetyl group from the intermediate acetyl-Iopamidol (VI),

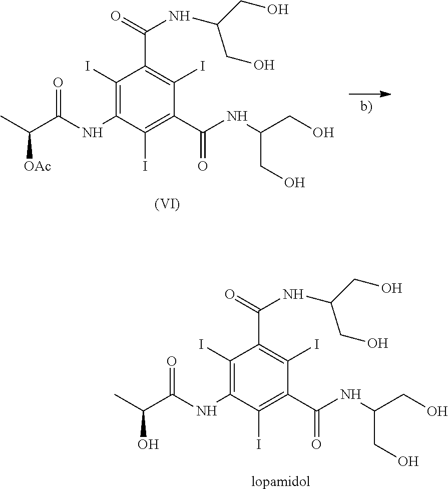

wherein said step a) of the process is carried out in continuous in a reactive extruder.

Preferably, the reaction of step a) is carried out at a temperature comprised in the range from 40° C. to 150° C. More preferably, said temperature is comprised in the range from 55° C. to 90° C.

In a preferred embodiment, the residence time of compounds (IV) and (V) in the reactive extruder is less than 15 minutes. More preferably, said residence time is comprised between 3 and 10 minutes.

The molar ratio between compound (V) and compound (IV) is preferably comprised between 3 and 15. More preferably, said molar ratio is comprised between 3 and 8.

In more detail, said step a) of the process defined above is carried out in a reactive extruder including at least one inlet section (A), a reactive chamber (B) and comprises the following steps:
i. continuously feeding the compounds (IV) and (V) into the at least one inlet section (A) of the reactive extruder;
ii. heating the reaction chamber (B) while mixing and transporting the reactant compounds (IV) and (V), thereby forming a product stream comprising a mixture of acetyl-Iopamidol (VI) and Iopamidol;
iii. collecting said product stream at the outlet section (C) of the reactive extruder.

In another aspect the present invention provides for a compound of formulation produced by the process of the invention.

Key extrusion parameters for successful formation of Iopamidol and/or acetyl-Iopamidol are mainly represented by the temperature and the residence time. In a preferred embodiment of the present invention the extrusion takes place under heating of the extruder.

Preferably the temperature of the reaction chamber (B) in step ii), and optionally of the at least one inlet section (A) in step i), is comprised in the range from 40° C. to 120° C. or from 55° C. to 90° C. or from 60° C. to 80° C. or from 70° C. to 80° C. More preferably it is comprised in the range from 55° C. to 90° C. Even more preferably, the temperature of the reaction chamber (B) is set at about 60° C. or 65° C. or 70° C. or 75° C. or 80° C. or 85° C. or 90° C.

In a preferred embodiment, to obtain a uniform temperature during the flow of the slurry, the reaction can be performed in an extruder having a plurality of heating means along the reaction chamber (B) and equipped with temperature measuring sensors, optionally suitable to also provide a temperature gradient or different temperature zones.

Preferably in the process of the invention the residence time of the slurry formed by compounds (IV) and (V) in the reaction chamber (B) is approximately less than 30 minutes; preferably the residence time is less than 15 minutes; more preferably it is comprised between 3 minutes and 10 minutes.

In a preferred embodiment the reaction of step a) is carried out in a reactive extruder selected from the group consisting of a single-screw extruder, a multi-screw extruder, such as twin-screw extruder, a vertical extruder, a planetary roller extruder and a ring extruder. More preferably, said reaction is carried out in a single-screw extruder or in a twin-screw extruder. In a preferred embodiment said twin-screw extruder has two co-rotating screws. In another embodiment said twin-screw extruder has two counter-rotating screws.

Preferably, the reactive extruder of the invention comprises at least one screw operating at a rotation speed comprised between 1 and 150 rpm.

The preferred molar ratio between serinol (V) and compound (IV) is comprised between 3 and 20 or between 3 and 15; more preferably said molar ratio is comprised between 3 and 8.

Based on the conditions applied, the acetyl group of compound (VI) tends to partially idrolyse so that the mixture obtained at the end of step a) can already comprise an amount of Iopamidol ranging between 0.5% and 20%. Preferably such amount ranges between 0.5% and 10%, more preferably between 0.5% and 5%. The remaining amount of acetyl-Iopamidol (VI) obtained from step a) is then hydrolysed during step b) to obtain the final Iopamidol.

The reactants for the process of the present invention can be prepared according to the methods described in the art. For instance, compound (IV) is a known intermediate in the synthesis of Iopamidol and can be prepared as disclosed in EP 2365963 B1 (example 4) while serinol (V) is a commercial product or can be prepared as described in EP 0348223 B1.

In the present description, and unless otherwise provided, the following terms and phrases as used herein are intended to have the following meanings.

The expression “residence time” refers to the average time required for the mixture of the reactants to pass through the extruder. Typically, such time depends on the length and pitch of the screw(s) and on their speed rotation and can last for a few minutes up to few hours. To avoid any possible degradation of the products it is important to calibrate the residence time also in view of the temperature control, since a product can be degraded during a short residence time at high temperature or during a longer residence time at lower temperature. The residence time can be determined experimentally, for instance by using a dye and measuring the time taken for the colored material to pass through the extruder.

The term “hopper” refers to a unit, typically shaped like a tapered cone, which is attached at a feed port and used to hold and feed the reactants into the extruder (inlet section A). In some instruments the hopper can be heated to keep the material hot or in a liquid state before melt processing. The one or more reactant(s) can be loaded in the hopper manually or therein conveyed using automatic systems.

The term “barrel” refers to a hollow chamber in which the screw(s) operates (reaction chamber B). It is generally made from thick alloy steel tubing or pipe in order to withstand the high pressures that can be generated within the extruder and can have different shapes depending on the configuration of the screw(s).

The reactive extruder suitable for the process of the invention can display different configurations. However, it is preferable that the extruder is equipped with heating means to provide for heating of the materials introduced into the reaction chamber.

Preferably the rotating screw(s) has a constant diameter, in particular close to the inlet section (A). Alternatively, and in particular in the central zone of the extruder, the screw(s) can have a continually increasing root diameter, which gradually increases the compressive forces on the reactants as they pass along the barrel.

In one embodiment the reactive extruder is a single screw extruder (SSE).

In another preferred embodiment the reactive extruder is a twin-screw extruder (TSE), having two screws that are modular and move in a mutually co-rotating or counter-rotating manner. The shear applied to the reactant material is a result of the enhanced mixing achieved by the interpenetration of the screws.

In a preferred embodiment at least one rotating screw is characterized by a continuous single segment with uniform pitch. Alternatively, the screw is characterized by profiles obtained by interchanging different screw segments with variable pitch, such as for instance kneading blocks (causing aggressive mixing), toothed segments (giving better dispersive mixing) or reverse segments (increasing the compressive forces and residence time). For instance, such rotating screws with variable pitch can be used with high viscosity materials, when a higher compressive force is needed.

It is a further aspect of the invention, the process as described above wherein step a) is carried out by further adding to the mixture at least one ingredient selected from a liquid additive and/or a base and/or an inert material.

In some embodiments, the reaction of step a) may further comprise the addition of a small amount of a liquid additive to the mixture of the reactants, in order to facilitate the sliding of the materials in case of high viscosity, increase their solubility and speed up the whole process. Preferably said liquid additive is added in the hopper of the inlet section (A) at the beginning of the process and together with at least one reactant. For example, it may be selected among safe and sustainable solvents, which are also easy to be removed at the end of the process without leaving any residue. For instance, in one embodiment such solvent is preferably selected from the group of glycerol, propylene glycol monomethyl ether (PGME), dipropylene glycol dimethyl ether (Proglyde), dipropylene glycol propyl ether, diethylene glycol diethyl ether, cyclopentyl methyl ether (CPME), gamma-valerolactone (GVL) and acetonitrile. The amount of liquid additive is preferably comprised between 0.1 and 10 equivalents. More preferably, the ratio of the equivalents between the compound (IV) and the liquid additive is comprised between 1:0.1 and 1:5. Even more preferably it is 1:0.5.

Alternatively, or simultaneously, a base can be added to the mixture of reactants in order to contribute to the neutralization of the reaction and to eventually reduce the total amount of serinol. Such base can be preferably an organic base selected among liquid materials in order to also act as a suitable lubricant contributing to a better mixing of the reactants. Examples of organic bases that can be used in the reaction of the invention are selected from potassium acetate and a tertiary amine, such as triethylamine, diisopropylethylamine (DIPEA), N-methylmorpholine, N-methylpiperidine or N-methylpyrrolidine. In another embodiment an inorganic base, for instance selected from Na₂CO₃, K₂CO₃, Cs₂CO₃, NaHCO₃, KHCO₃, CaO and the like, can be used. Preferably, the process of the invention is carried out by using from 0.1 to 10 equivalents of additional base for each equivalent of compound (IV) undergoing amidation. More preferably, the process is carried out by using 5 equivalents of additional base for each equivalent of compound (IV).

In other embodiments, the reaction of step a) may further comprise the addition of an inert material to the mixture of the reactants, in order to facilitate the mechanochemical effect and foster the mixing of the reactants. For instance, an immiscible material like NaCl or Na₂SO₄ can be added to the mixture of serinol (V) and compound (IV) before their introduction in the extruder, preferably in an amount comprised between 10 and 30 weight %.

The reactants, optionally together with a base and/or a liquid additive and/or an inert material, can be continuously fed into the reaction chamber either simultaneously or in different steps. Moreover, reactants can be fed into one single inlet section (A), optionally after having been pre-mixed in a separate container, or they can be fed separately into different inlet sections, respectively (A′) and (A″).

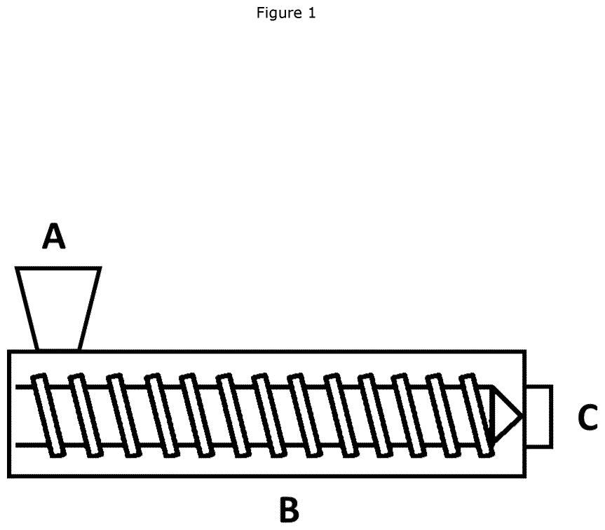

A typical configuration of a reactive extruder suitable for the process of the present invention is illustrated in FIG. 1, wherein the reaction chamber (B) consists of a single screw system which is continuously fed with compounds (IV) and (V) through a unique inlet section (A). Preferably, the compounds (IV) and (V) can be pre-mixed in a preconditioning step before being loaded at once or portionwise in the inlet section (A), at room temperature or optionally heated at a temperature of 30° C. or higher. Such pre-mixing allows to obtain an optimal blending of the materials and to form a slurry suitable to be continuously supplied into the reactive extruder.

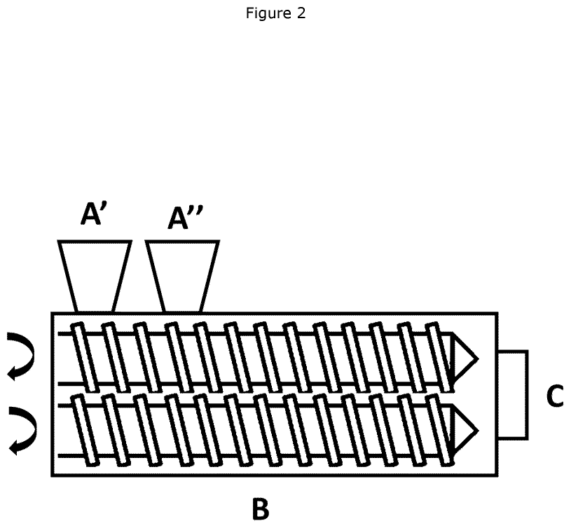

Another preferred configuration of a reactive extruder suitable for the process of the present invention is illustrated in FIG. 2, wherein the reaction chamber (B) consists of a co-rotating twin-screws system which is continuously fed with compounds (IV) and (V) through separate inlet sections (A′) and (A″).

Said inlet sections (A) or (A′) and (A″) typically consist of a hopper of different possible forms and are positioned upstream, at the beginning of the reaction chamber. In case of two inlet sections (A′) and (A″), these hoppers are configured so that the inlet (A′) is used for adding serinol (V) and is located upstream, whilst the inlet (A″) is used for adding the compound (IV) and is located downstream with respect to (A′). Alternatively, (A′) and (A″) can be located in different points of the same cross-section of the barrel. In one embodiment the extruder with separate independent feeding means can be also configured with a first mixing zone in correspondence to the inlet section(s).

Preferably, said inlet sections are able to provide heating to the materials introduced into the reaction chamber. For instance, in one embodiment the inlet section for the feeding of serinol (V) is heated at a temperature higher than 50° C. so that it is melted and then mixed in the reaction chamber when compound (IV) is fed as a solid. Preferably in this case compound (V) is fed in a pre-heated upstream inlet (A′) while compound (IV) is fed in the downstream or parallel inlet (A″) which can be optionally heated or not heated.

Generally, the one or more screws as described above are characterized by a cylindrical or a conical shape, with a diameter comprised in the range of 5-35 mm, and are operated at a rotation speed comprised in the range between 0.5 and 40 rpm.

In certain embodiments of the invention, the extruder can be also equipped with a degassing port for venting any possible gas formed during the extrusion process.

Another parameter that can affect the efficiency of the extrusion process is represented by the size distribution of the solid materials fed into the reactive chamber, in particular of compound (IV). In one embodiment the reactants, even when fed separately or after pre-mixing, are preferably grinded, e.g. in a mortar, before being loaded in the inlet section(s), in order to reduce the particle size of the solid material to a value below 1 mm. For instance, a powder of compound (IV) with a small particle size can be accomplished by using a micronizing instrument or a planetary ball mill with a mix of balls of different size rotating at a suitable speed.

Generally, the reactants are fed into the inlet section(s) at a feed rate comprised between 5 and 30 g/min, depending on the screw rotation speed. Such rotation speed is preferably set between 1 and 150 rpm, but more preferably the instrument is operated with a rotation speed from 0.5 to 40 rpm. For instance, the reactants are fed at a feed rate of 5 g/min when the screw rotation speed is 5 rpm and at a feed rate of 30 g/min when the screw rotation speed is 30 rpm.

In one embodiment, the reaction extrusion may be possibly combined with on-line monitoring via e.g. Raman or infrared spectroscopy, which can provide information about the chemical reactions taking place inside the extruder.

After extrusion, the extrudate mixture is collected at the outlet section (C). In some embodiments such extrudate is represented by a pasty material which can be recovered in a container for further treatment.

According to the process of the invention the conversion of the residual amount of intermediate compound (VI) into Iopamidol (step b) is carried out under basic aqueous conditions, for instance as described in EP2365963 B1 (example 3).

The pasty extrudate can be directly collected at the outlet (C) in a separate recipient comprising an aqueous solution, and conveniently treated by addition of an aqueous basic solution such as diluted sodium hydroxide (e.g. NaOH 30% wt.) up to a pH of about 10, and heating at a temperature ranging between 25° C. and 50° C. for at least 1 hour up to 10 hours, preferably for 7 hours. As alternative, the hydrolysis can be also performed by means of a basic exchange resin, according to known methods.

The reaction crude thus obtained may be then neutralized to a pH of 6-7 with HCl, and purified by ion exchange resins according to the procedures known in the art, e.g. as disclosed in EP2365963 B1, or any other suitable method of purification (e.g. desalination with electrodialysis or ion exchange resins, distillation, chromatography, crystallization).

The Iopamidol product obtained with the process of the invention may be used to prepare a formulation, e.g. for use in a subject as X-rays contrast agent, by dissolving it in an aqueous solution.

The technical solution provided by the present invention, represented by the use of the reactive extrusion of intermediate reactants for manufacturing Iopamidol, is of general applicability and provides a synthetic approach generally exploitable for the preparation of radiographic contrast agents and/or their key intermediates.

Accordingly, in an additional embodiment the invention relates to a process for the synthetic preparation of a radiographic intermediate of formula (VII)

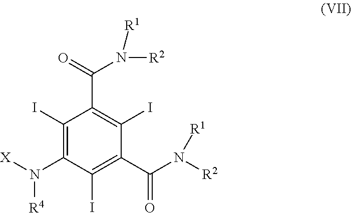

wherein
R¹ is a C₁-C₆-alkyl substituted by one or more hydroxyl groups;
R² is hydrogen or a C₁-C₆-alkyl optionally substituted by one or more hydroxyl groups;
X is hydrogen or a group —COR³ wherein
R³ is a C₁-C₆-alkyl optionally substituted by one or more a hydroxy, C₁-C₄-alkoxy or acetyloxy (—OAc) groups,
R⁴ is hydrogen or C₁-C₆-alkyl,
   which comprises:
c) obtaining a 5-amino-2,4,6-triiodo-1,2-benzenedicarboxylic acid dichloride of formula (VIII), wherein X and R⁴ are as defined above

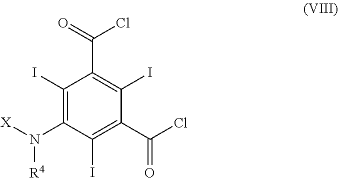

and
d) reacting the obtained dichloride with an amine of formula NHR¹R², wherein R¹ and R² are defined above,
wherein said reaction step d) is carried out in continuous by means of a reactive extrusion process, according to the solution identified by the present invention.

Further details concerning the manufacturing of Iopamidol according to the present invention are reported in the following experimental section, with the sole aim to better illustrate the present invention, without representing any limitation to it.

### EXPERIMENTAL PART

### Materials and Methods

All commercially available reagents and solvents were used without further purification. 2-amino-1,3-propanediol (serinol, V) was purchased from TCI Chemicals and the intermediate compound (IV) was prepared substantially as described in EP 2365963 B1.

The reactive extrusion was performed using different extrusion instruments. For instance, the equipment was selected among the following: i) a conical twin-screw extruder (Mod. REM-2CA, Zamak Mercator) equipped with co-rotating twin-screws in a reaction chamber having a capacity from 5 to 20 mL; ii) a conical twin-screw Extruder (Mod. MiniLab II HAAKE Rheomer CTW5, Termo Fischer Scientific) equipped with conical screws having diameter of 5/14 mm and length of 109.5 mm; iii) a single screw extruder (Mod. TR12/20GM manufactured by Gimac, Italy) equipped with a single feed, four independently controlled temperature zones and a 35 cm long screw with constant pitch.

### Analytical Characterization

Analytical characterization of the compounds was performed by capillary electrophoresis MEKC-CE, RP-HPLC or MS, using the following conditions:

### MEKC-CE Method

Instrument: Agilent HP G1600AX 3D Capillary Electrophoresis System
Capillary: silica, 65 cm, 50 micron ID
Inlet=Outlet=running buffer: Na₂B₄O₇ 25 mM+SDS 20 mM
Injection: 50 mbar×3″ sample+30 mbar×2″ running buffer
Temperature: 45° C.
Voltage: +25 kV
UV detection: 227-240 nm

### RP-HPLC Method

Column: Zorbax SB Phenyl 80 Å, 5 μm 250×4.6 mm
Flow rate: 2 mL/min
Injection volume: 20 μL
UV detector: spectrum recording between 200 and 400 nm; analysis at 254 nm
Mobile phase: Eluent A: water, Eluent B: acetonitrile/water 1:1

Gradient:

| Time (min) | % Eluent A | % Eluent B |
|---|---|---|
| 0 | 100 | 0 |
| 9 | 100 | 0 |
| 13 | 62 | 38 |
| 16 | 50 | 50 |
| 18 | 100 | 0 |
| 22 | 100 | 0 |

### Quantitative MS Method

Instrument: Waters Micromass ZQ
Ionization source: ESI+
Cone: 15 V
M/z range: 90-300

### Example 1—Preparation of Iopamidol in a Twin-Screw Extruder

Serinol (V) (99.5 g; 1.09 mol) and compound (IV) (77.51 g; 0.109 mol), prepared as described in EP2365963 were pre-treated by grinding in a mortar (molar ratio of compound (IV) vs serinol (V) was 1:10).

The mixture of the reactants was then fed into a continuously operated twin-screw extruder maintained at a temperature of 100° C. and atmospheric pressure. The mixture was continuously charged by in the inlet system (A) during a loading time of 16 minutes.

The reaction was carried out in continuous with an average residence time in the apparatus of 5 minutes. Samples of about 10-20 g of pasty mixture were collected every 2 minutes at the outlet into separate vessels. After 28 minutes the extruder was stopped, with a total collection of 118 g of final mixture. Each sample of extrudate thus obtained (10-20 g) was dissolved in 500 mL of a basic aqueous solution containing 7.5 g of KOH and kept for 15 minutes in ultrasonic bath at room temperature to complete the hydrolysis of the remaining acetyl-Iopamidol. 5 mL of the obtained solution were diluted 1:1 with water and analysed by MECK-CE, after adding 60-100 mg of benzamide to each sample as internal standard.

The conversion of compound (IV) was obtained in a range from 70% to 85%, calculated from the MECK-CE peak area %.

The yield of Iopamidol in each sample after the hydrolysis was comprised in the range from 60% to 70%.

### Example 2—Preparation of Iopamidol in a Single-Screw Extruder

9.0 g of Compound (IV) (0.013 mol) were grinded in a Retsch PM100 planetary ball mill for 2 minutes at 350 rpm and subsequently pre-mixed in a recipient with 6.0 g of serinol (V) (0.066 mol) (molar ratio of compound (IV) vs serinol (V) was 1:5).

The mixture of the reactants was then fed simultaneously into a continuous single-screw extruder with 4 different temperature zones: two zones respectively next to the inlet and outlet sections were maintained at 50° C., while the two central zones were maintained at a temperature of 70° C. (atmospheric pressure). The mixture was continuously charged in the inlet system (A) during a feeding time of 6 minutes.

The reaction was carried out in continuous with an average residence time in the apparatus of 5.5 minutes. After 1 minute the extruder was stopped, with a total collection of 2.7 g of final mixture. The extrudate thus obtained was then directly dissolved in 2.0 mL of 20% NaOH aqueous solution at room temperature and stirred for 1 hour to complete the hydrolysis of the remaining acetyl-Iopamidol and conversion to Iopamidol.

The basic solution was then neutralized with 2N HCl. The yield of the derived Iopamidol was determined by RP-HPLC analysis of the crude solution by use of an external standard.

The conversion of compound (IV) was 96%, calculated from the HPLC peak area %.

The yield of Iopamidol after the hydrolysis was 74%.

### Example 3—Preparation of Iopamidol in a Single-Screw Extruder in Presence of a Liquid Additive

9.0 g of Compound (IV) (1 eq, 0.013 mol) were pre-mixed in a recipient with 6.0 g of serinol (V) (5 eq, 0.066 mol), then 1.2 mL of DPnP (0.5 eq, 0.007 mol) were added dropwise.

The mixture of the reactants was fed manually in the inlet system (A) of a single-screw extruder with 4 different temperature zones: two zones respectively next to the inlet and outlet sections were maintained at 50° C., while the two central zones were maintained at a temperature of 70° C. (atmospheric pressure). The reaction was carried out in continuous with a residence time in the apparatus of 8 minutes.

The extrudate was then directly collected into 50 mL vials containing 2.0 mL of 20% NaOH aqueous solution at room temperature; the vials were stirred for 1 hour to carry out the hydrolysis of acetyl-Iopamidol and obtain Iopamidol.

The basic solution was then neutralized with 2N HCl. The yield of Iopamidol was determined by RP-HPLC analysis of the crude solution by use of an external standard.

The conversion of compound (IV) was 81.9%, calculated from the HPLC peak area %.

The yield of Iopamidol after the hydrolysis was 69.7%.

### Example 4—Preparation of Iopamidol in a Single-Screw Extruder in Presence of a Base

9.0 g of Compound (IV) (1 eq, 0.013 mol) were pre-mixed in a recipient with 6.0 g of serinol (V) (5 eq, 0.066 mol) together with 8.8 g of K₂CO₃ (5 eq, 0.064 mol).

The mixture of the reactants was fed manually in the inlet system (A) of a single-screw extruder with 4 different temperature zones: two zones respectively next to the inlet and outlet sections were maintained at 50° C., while the two central zones were maintained at a temperature of 70° C. (atmospheric pressure). The reaction was carried out in continuous with a residence time in the apparatus of 8 minutes.

The extrudate was then directly collected into 50 mL vials containing 2.0 mL of 20% NaOH aqueous solution at room temperature which were stirred for 1 hour to carry out the hydrolysis of acetyl-Iopamidol and obtain Iopamidol.

The basic solution was then neutralized with 2N HCl. The yield of Iopamidol was determined by RP-HPLC analysis of the crude solution by use of an external standard.

The conversion of compound (IV) was 81.8%, calculated from the HPLC peak area %.

The yield of Iopamidol after the hydrolysis was 66.2%.

### Example 5—Preparation of Iopamidol in a Single-Screw Extruder in Presence of an Inert Ingredient

9.0 g of Compound (IV) (1 eq, 0.013 mol) were pre-mixed in a recipient with 6.0 g of serinol (V) (5 eq, 0.066 mol) together with 2.6 g of NaCl (15% w/w, 0.044 mol).

The mixture of the reactants was fed manually in the inlet system (A) of a single-screw extruder with 4 different temperature zones: two zones respectively next to the inlet and outlet sections were maintained at 50° C., while the two central zones were maintained at a temperature of 70° C. (atmospheric pressure). The reaction was carried out in continuous with a residence time in the apparatus of 8 minutes.

The extrudate was then directly collected into 50 mL vials containing 2.0 mL of 20% NaOH aqueous solution at room temperature which were stirred for 1 hour to carry out the hydrolysis of acetyl-Iopamidol and obtain Iopamidol.

The basic solution was then neutralized with 2N HCl. The yield of Iopamidol was determined by RP-HPLC analysis of the crude solution by use of an external standard.

The conversion of compound (IV) was 81.4%, calculated from the HPLC peak area %.

The yield of Iopamidol after the hydrolysis was 71.8%.

### REFERENCES

1. The Merck Index, RSC Publishing, 15^{th} Ed., 2013, 940-941
2. Lusic, H. et al., Chem. Rev. 2013, 113, 1641-1666
3. GB 1,472,050
4. U.S. Pat. No. 4,001,323
5. GB 2,311,524
6. WO00/15602
7. WO2018/104228
8. Crawford D. E. et al, Green Chem. 2017, 19, 1507
9. Crawford D. E. et al, Chem. Commun. 2017, 53, 13067-13070
10. Lavayssiere M. et al, Chem. Commun. 2023, 59, 3439-3442
11. U.S. Pat. No. 5,859,269
12. U.S. Pat. No. 5,859,263
13. U.S. Pat. No. 9,453,107
14. EP 2365963
15. EP 0348223

1. A process for the preparation of N¹,N³-bis[2-hydroxy-1-(hydroxymethyl)ethyl]-5-[[(2S)-2-hydroxy-1-oxopropyl]amino]-2,4,6-triiodo-1,3-benzenedicarboxamide (Iopamidol) comprising the following steps:
a) mixing and reacting S-5-[[2-(acetyloxy)-1-oxopropyl]amino]-2,4,6-triiodo-1,3-benzenedicarboxylic acid dichloride (IV) with 2-amino-1,3-propanediol (V) thus obtaining a mixture of the intermediate acetyl-Iopamidol (VI) and Iopamidol 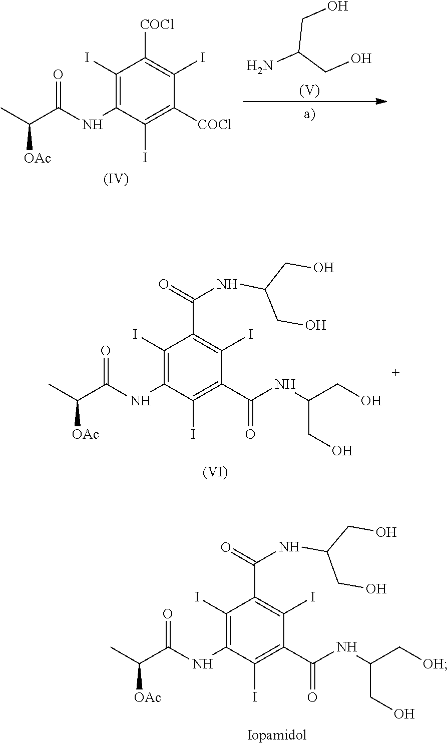
b) treating the mixture thus obtained in basic aqueous conditions by promoting the removal of the acetyl group from the intermediate acetyl-iopamidol (VI) 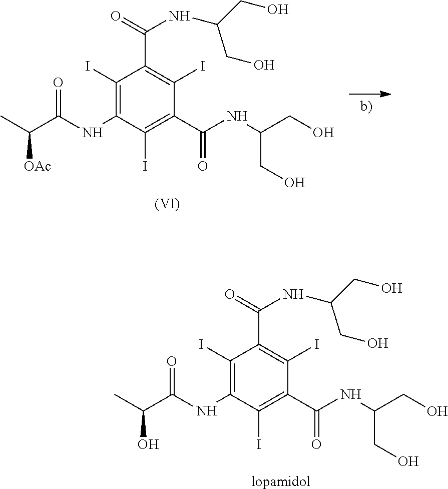
wherein said step a) of the process is carried out in continuous in a reactive extruder.

2. The process according to claim 1, wherein the reaction of step a) is carried out at a temperature comprised in the range from 40° C. to 150° C.

3. The process of claim 2, wherein said temperature is comprised in the range from 55° C. to 90° C.

4. The process according to claim 1 wherein the residence time of compounds (IV) and (V) in the reactive extruder is less than 15 minutes.

5. The process according to claim 1 wherein the residence time of compounds (IV) and (V) in the reactive extruder is comprised between 3 and 10 minutes.

6. The process according to claim 1 wherein the molar ratio between compound (V) and compound (IV) is comprised between 3 and 15.

7. The process according to claim 6 wherein the molar ratio between compound (V) and compound (IV) is comprised between 3 and 8.

8. The process of claim 1 wherein said reactive extruder includes at least one inlet section (A), a reactive chamber (B) and an outlet section (C), and step a) comprises the following steps:
i. continuously feeding the compounds (IV) and (V) into the at least one inlet section (A) of the reactive extruder;
ii. heating the reaction chamber (B) while mixing and transporting the reactant compounds (IV) and (V), thereby forming a product stream comprising a mixture of acetyl Iopamidol (VI) and Iopamidol;
iii. collecting said product stream at the outlet section (C) of the reactive extruder.

9. The process according to claim 1 wherein the reactive extruder is selected from the group consisting of single-screw extruder, multi-screw extruder, twin-screw extruder, vertical extruder, planetary roller extruder and ring extruder.

10. The process according to claim 1 wherein the reactive extruder is a single screw extruder.

11. The process according to claim 1 wherein the reactive extruder is a twin-screw extruder.

12. The process according to claim 1 wherein the reactive extruder comprises at least one screw operating at a rotation speed comprised between 1 and 150 rpm.

13. The process according to claim 1 wherein compounds (IV) and (V) in step a) are pre-mixed and said mixture is continuously fed into the inlet section (A).

14. The process according to claim 1 wherein compounds (IV) and (V) in step a) are fed separately into independent inlet sections (A′) and (A″).

15. The process according to claim 1 wherein step a) is carried out by further adding to the mixture at least one ingredient selected from a liquid additive and/or a base and/or an inert material.

16. The process according to claim 15 wherein said liquid additive is selected from glycerol, propylene glycol monomethyl ether (PGME), dipropylene glycol dimethyl ether (Proglyde), dipropylene glycol propyl ether, diethylene glycol diethyl ether, cyclopentyl methyl ether (CPME), gamma-valerolactone (GVL) and acetonitrile.

17. The process according to claim 15 wherein said base is selected from potassium acetate; a tertiary amine, triethylamine, diisopropylethylamine (DIPEA), N-methylmorpholine, N-methylpiperidine, N-methylpyrrolidine; an inorganic base, Na₂CO₃, K₂CO₃, Cs₂CO₃, NaHCO₃, KHCO₃ and CaO.

18. The process according to claim 15 wherein said inert material is selected from NaCl and Na₂SO₄.

19. The process according to claim 1 wherein the conversion of step b) is carried out by adding the pasty material collected from step a) into an aqueous basic solution and heating at a temperature ranging from 25° C. to 50° C. for at least 1 hour.

20. A process for the synthetic preparation of a radiographic intermediate of formula (VII) 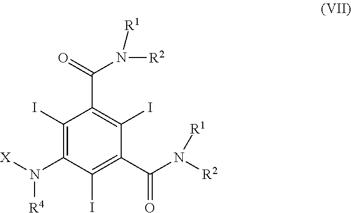
wherein
R¹ is a C₁-C₆-alkyl substituted by one or more hydroxyl groups;
R² is hydrogen or a C₁-C₆-alkyl optionally substituted by one or more hydroxyl groups;
X is hydrogen or a group —COR³ wherein
R³ is a C₁-C₆-alkyl optionally substituted by one or more a C₁-C₄-alkoxy or acetyloxy (—OAc) groups,
R⁴ is hydrogen or C₁—C-alkyl,
which comprises:
c) obtaining a 5-amino-2,4,6-triiodo-1,2-benzenedicarboxylic acid dichloride of formula (VIII), wherein X and R⁴ are as defined above 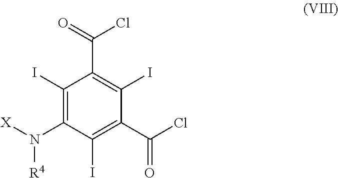
and
d) reacting the obtained dichloride derivative (VIII) with an amine NHR¹R², wherein R¹ and R² are defined above,
wherein said reaction of step d) is carried out in continuous by means of a reactive extrusion process.
